(19) United States
(12) Patent Application Publication
(10) Pub. No.: US 2009/0124585 A1
(43) Pub. Date: May 14, 2009

(54) Novel Crystalline Pharmaceutical Product

(76) Inventors: Wendy Isabel Cross, Hertfordshire (GB); Matthew Lawrence Hannan, Hertfordshire (GB); David Michael Johns, Hertfordshire (GB); Mei-yin Lee, Hertfordshire (GB); Christopher John Price, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/910,615

(22) PCT Filed: Apr. 6, 2006
(86) PCT No.: PCT/EP2006/003197

§ 371 Date: Oct. 4, 2007

(30) Foreign Application Priority Data
Apr. 8, 2005 (GB) 0507165.9

(51) Int. Cl. A61K 31/58; C07J 17/00

(52) U.S. Cl. 514/172; 540/114

(57) ABSTRACT

There are provided crystalline particles of unsolvated Form 1 polymorph of the compound of formula (I):

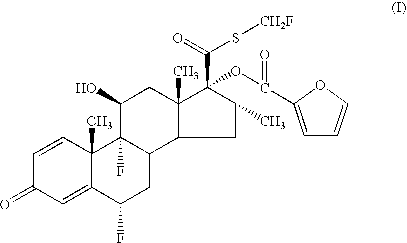

characterised in that the particles are in the form of substantially triangular plates.

The present invention relates to a novel crystalline habit of a glucocorticoid and to processes for its preparation. The present invention also relates to pharmaceutical formulations containing the crystalline product and to therapeutic uses thereof, particularly for the treatment of inflammatory and allergic diseases.

Glucocorticoids which have anti-inflammatory properties are known and are widely used for the treatment of inflammatory disorders or diseases such as asthma and rhinitis. For example, U.S. Pat. No. 4,335,121 discloses 6α,9α-difluoro-17α-(1-oxopropoxy)-11β-hydroxy-16α-methyl-3-oxo-androsta-1,4-diene-17β-carbothioic acid S-fluoromethyl ester (known by the generic name of fluticasone propionate) and derivatives thereof. The use of glucocorticoids generally, and especially in children, has been limited in some quarters by concerns over potential side effects. The side effects that are feared with glucocorticoids include suppression of the Hypothalamic-Pituitary-Adrenal (HPA) axis, effects on bone growth in children and on bone density in the elderly, ocular complications (cataract formation and glaucoma) and skin atrophy. Certain glucocorticoid compounds also have complex paths of metabolism wherein the production of active metabolites may make the pharmacodynamics and pharmacokinetics of such compounds difficult to understand. Whilst the modern steroids are very much safer than those originally introduced, it remains an object of research to produce new molecules which have excellent anti-inflammatory properties, with predictable pharmacokinetic and pharmacodynamic properties, with an attractive side effect profile, and with a convenient treatment regime.

International Patent Application WO02/12265 discloses a novel glucocorticoid compound which substantially meets these objectives namely 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

In the field of inhalation therapy, particles comprised of therapeutic molecules are generally desired of a particle size “suitable for inhalation”, which is a term generally taken to indicate an aerodynamic diameter between 1 and 10 μm, especially between 1 and 5 μm, particularly between 1 and 3 μm.

Particles of the desired particle size for inhalation therapy are conventionally prepared by milling or micronisation. These processes, depending on the precise conditions adopted, are capable of generating particle distributions which include fractions having particles with the appropriate size. However, there are a number of disadvantages associated with milling and micronisation processes including that the fraction having the desired particle size may be relatively small, that there may be generated a significant fraction of particles that are finer than is desired (which may be deleterious e.g. if it affects bioavailability) and that product losses generally may be considerable (e.g. through coating of the machinery). A further property of micronised products is that the surfaces of the particles generated may be substantially amorphous (i.e. have minimal crystallinity). This may be undesirable when there exists a tendency for the amorphous regions to convert to a more stable crystalline state. Furthermore micronised or milled products may be more susceptible to moisture uptake than crystalline products. Micronisation and milling processes also suffer from the disadvantages that they are relatively energy intensive and require containment and other measures to avoid the risk of dust explosion.

The formation of crystalline particles of the desired size by rapid precipitation (e.g. by dilution of a solution with an anti-solvent) may give rise to particles of suitable size. Control of the habit and size of crystals produced according to such processes is a valuable tool in adjusting and optimising pharmaceutical and biological properties such as flow characteristics, aerodynamic properties, dissolution rate and bioavailability.

The glucocorticoid compound 6α,9α-difluoro-17α-[(2-furanylcarbonyl)oxy]-11β-hydroxy-16α-methyl-3-oxo-androsta-1,4-diene-17β-carbothioic acid S-fluoromethyl ester exists in a number of different solid state forms. In unsolvated form it has been found to exist in 3 crystalline polymorphic forms, Forms 1, 2 and 3, and these polymorphic forms are characterised by their XRPD patterns as described in WO03/066656.

Broadly speaking the Forms are characterised in their XRPD profiles as follows:

Form 1: Peak at around 18.9 degrees 2Theta

Form 2: Peaks at around 18.4 and 21.5 degrees 2Theta.

Form 3: Peaks at around 18.6 and 19.2 degrees 2Theta.

Processes for the production of crystalline forms of this compound are described in WO02/12265 and WO03/066656. Crystalline unsolvated Form 1 polymorph may be produced by dissolving the compound in methyl isobutyl ketone or ethyl acetate and adding an anti-solvent such as iso-octane or toluene. Alternatively the compound may be dissolved in methyl-isobutyl-ketone and iso-octane added as anti-solvent. These processes as described in WO03/066656 give rise to needle shaped crystals.

Crystalline unsolvated Form 1 polymorph may be also be prepared from the crystalline complexes described in WO03/066656. Equant or substantially equant particles (typically elongated tetragonal bipyramidal crystals) of the complexes with the guest molecule acetone or propan-2-ol may be converted to unsolvated Form 1 polymorph by removal of the guest molecule, for example, by heating to around 100-110° C. Unsolvated polymorph Form 1 when prepared by this method is produced in the form of equant or substantially equant particles. These crystals are more readily micronised than the needle shaped crystals prepared by the methods described above involving e.g. recrystallization from ethylacetate and toluene.

Equant and substantially equant particles may be single crystals or agglomerations of crystals. Equant particles have dimensions in each of the three axes of measurement which are approximately the same, for example they have dimensions in the three axes such that the difference between the largest and the smallest measurement is not more than approximately 50% of the smallest. Particles which are single crystals are typically equant. Particles which are agglomerations of crystals are typically substantially equant such that the particles have dimensions in the three axes such that the difference between the largest and the smallest measurement is not more than approximately 100% of the smallest, particularly not more than 50% of the smallest.

It is therefore desirable to produce unsolvated Form 1 polymorph of the compound 6α,9α-difluoro-17α-[(2-furanylcarbonyl)oxy]-11β-hydroxy-16α-methyl-3-oxo-androsta-1,4-diene-17β-carbothioic acid S-fluoromethyl of crystal habit showing improved properties over the crystal habits produced using the methods described above. In particular it is desirable to produce crystal habits showing good mechanical strength, handling and aerodynamic properties. Furthermore it is desirable to produce crystals of respirable or near respirable aerodynamic size directly, eliminating the requirement for micronisation.

We have now identified a novel crystalline habit of the glucocorticoid compound 6α,9α-difluoro-17α-[(2-furanylcarbonyl)oxy]-11β-hydroxy-16α-methyl-3-oxo-androsta-1,4-diene-17β-carbothioic acid S-fluoromethyl which has surprising advantages over crystalline forms described previously.

Thus, according to a first aspect of the invention, there are provided crystalline particles of the unsolvated Form 1 polymorph of the compound of formula (I):

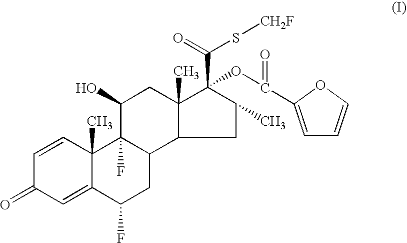

characterised in that the particles are in the form of substantially triangular plates (hereinafter the “crystalline particles of the invention”).

According to a further aspect of the invention, there are provided crystalline particles of the unsolvated Form 1 polymorph of the compound of formula (I):

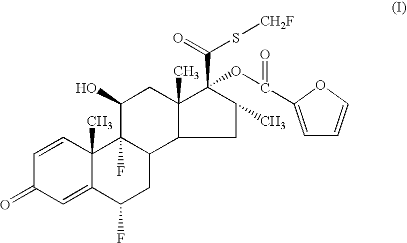

characterised in that the particles are in the form of triangular plates.

The chemical name of the compound of formula (I) is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

The crystalline particles of unsolvated Form 1 polymorph of the compound of formula (I) is characterised in that the particles are of space group P2₁ and have cell dimensions of 7.6, 14.1, 11.8 Å when determined at 150K. Space group P2₁ is characterised by having two axis angles of 90°.

The particles of the invention have a triangular plate crystal habit, typically a substantially isosceles triangular plate habit. Typically the particles have 5 faces: two faces which are triangular and three which are rectangular. Typically the angles of the triangular faces are approximately 80°, 50° and 50°. The particles of the invention have orthogonal dimensions in two of the three axes of measurement which are approximately the same, e.g. the larger dimension is not more than twice the smaller dimension, preferably not more than 75% greater than the smaller dimension; and a dimension in the third orthogonal axis of measurement which is less than one fifth, e.g. approximately one tenth, that of the next smallest dimension. For example in the case of a triangular plate having triangular faces with angles of approximately 80°, 50° and 50° the largest dimension is approximately 1.5 times that of the next largest orthogonal dimension. The particles of the invention are, for example, of size 0.1-0.2 μm×4-5 μm×4-5 μm.

The term substantially triangular includes plates where one or more corners of the triangle are truncated.

The crystal habit of the particles of the invention can be seen by reference to FIGS. 1 to 3 and 6.

As shown in FIG. 4, an XRPD profile of the particles of the invention when crystallographically pure exhibits a peak at around 18.9 degrees 2Theta which is characteristic of Form 1.

The particles of the invention may be prepared by the methodology described hereinafter, which constitutes a further aspect of this invention.

A process for preparing the particles of the invention comprises crystallising the compound of formula (I).

Thus according to the invention there is provided a process for preparing a crystalline unsolvated Form 1 polymorph of the compound of formula (I):

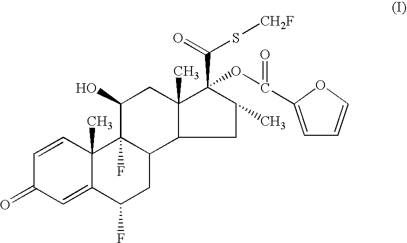

wherein the particles are in the form of triangular plates, which process comprises dissolving the compound of formula (I) in a solvent of methyl-isobutyl-ketone (MIBK) containing between 1% and 15% v/v methyl-ethyl-ketone (MEK), and producing compound of formula (I) as unsolvated Form 1 polymorph by addition of heptane as anti-solvent.

The proportion of MEK in the MIBK/MEK feed solvent mixture should be as high as possible to enhance the solubility of the compound of formula (I) in the solvent mixture, but not so high as to result in the crystalline product so produced being in the form of an MEK solvate. Enhancing the solubility of the compound of formula (I) in the solvent mixture provides processing advantages as it reduces the volume of solvent which is required. We prefer the solvent to contain >5% v/v MEK.

Preferably the process comprises dissolving the compound of formula (I) in a solvent of methyl-isobutyl-ketone (MIBK) containing between 8% and 11% especially around 10% v/v methyl-ethyl-ketone (MEK). In this range the process is efficient without undue risk of forming the MEK solvate. Also any risk of encrustation in the crystallizers is reduced.

For reasons of operational efficiency the process is ideally operated at a temperature of between 10 and 40° C.

The input compound of formula (I) used to produce the MIBK:MEK solution for use in the process of the invention is preferably relatively pure, typically greater than 95% pure and preferably greater than 97% pure.

The compound of formula (I) may be prepared by alkylation of the corresponding thioacid, or a salt thereof, as described in WO02/12265.

The MIBK/MEK solvent and heptane anti-solvent system described above produces crystalline product of particularly high purity and as such may, when different processing conditions are used, be useful for the production of crystalline unsolvated Form 1 polymorph of the compound of formula (I) having crystal habits other than triangular plates. Thus according to a further aspect of the invention there is provided a process for preparing a crystalline unsolvated Form 1 polymorph of the compound of formula (I):

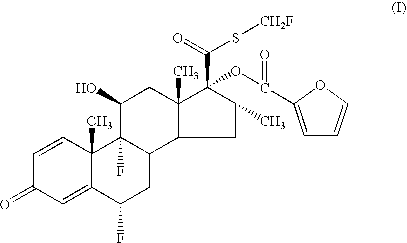

which process comprises dissolving the compound of formula (I) in a solvent mixture of methyl-isobutyl-ketone (MIBK) containing between 8 and 11% v/v methyl-ethyl-ketone (MEK) and producing compound of formula (I) as unsolvated Form 1 polymorph by addition of heptane as anti-solvent.

The particles of the invention are preferably prepared in a continuous process e.g. using a process which comprises mixing in a vessel (or more than one vessel) a flowing solution of compound of formula (I) in MIBK and MEK with flowing heptane as anti-solvent. Preferably the process is performed in the presence of ultrasound radiation.

It is desirable to mix the solution with the antisolvent in the presence of ultrasound radiation since this increases the nucleation rate and enhances the ability to produce small particles.

Desirably the flow cell will include a stirrer.

The crystallization is preferably performed in a continuous manner with a residence time of more than 20 mins, typically within the range 40 to 360 minutes for an almost saturated solution of composition 9:1 v/v MIBK:MEK.

Residence time is the time taken for the crystallizer (or all of them if more than one is employed) to fill from empty to the operating level when fed with the solution of drug substance dissolved in MEK/MIBK of the selected ratio and with the corresponding heptane anti solvent flow rate.

Circumstances of too high supersaturation, which may arise in a batch process or a continuous process with too high a rate of addition of antisolvent to solution, should generally be avoided as these may lead to undesired crystal elongation and agglomeration.

If a residence time of less than 20 minutes is employed this tends to result in undesired elongation of the crystals growing at too high a supersaturation. Preferably the residence time is greater than 60 minutes e.g. around 80 to 160 minutes.

The particles of the invention are preferably prepared in a continuous flow manner using a “multiple crystallizer”, e.g. a twin crystallizer as shown in FIG. 5. In a multiple crystallizer the outflow from a first continuous flow cell is transferred to a second (and optionally subsequent) continuous flow cell before collecting the particles outflowing from the final flow cell. Each additional crystallizer after the first has a heptane anti-solvent feed

Operating with multiple e.g. two crystallizers in sequence is advantageous, reducing the tendency to encrust by allowing generation of supersaturation in each crystallizer vessel rather than generating all the supersaturation in the first vessel.

It may be preferred to operate the individual crystallizers at different temperatures e.g. to operate the first crystallizer at a higher temperature than the second crystallizer. For example the first crystallizer may be operated at a temperature of around 30° C. and the second crystallizer may be operated at a temperature of around 10° C.

The heptane flow rates to each of the crystallizers may be adjusted to control the amount of crystallization taking place in each vessel limiting the supersaturation to reduce the risk of encrustation.

The crystallization process described here benefits from seeding with particles of the invention to initiate it in order to reduce the risk of encrustation forming in the initial stages of an unseeded crystallization when supersaturation is higher than would be achieved in a seeded crystallization.

Several start up strategies may be adopted:

The crystallizer(s) may first be charged with a solvent composition which matches that which will be achieved during steady state operation (excluding the contribution of the compound of formula (I)). This solvent mixture in the crystallizer vessel(s) is then slowly displaced as the feed solution of the compound of formula (I) and the heptane antisolvent flow into the crystallizers.

Alternatively the crystallizer(s) can be charged by initiating the feed flows at the selected rates for the experiments and filling the crystallizer vessel(s) from empty.

Alternatively the crystallizer(s) can be charged from empty by initiating the feed flows at higher rates than those selected for the steady state operation of the crystallization system, reverting to the selected rates for the experiments once the crystallizer vessels are filled to the operating level.

Whichever start up strategy is selected the ultrasound equipment (if employed) may be switched on during the start up phase of the crystallization experiment when the ultrasound horns are partially immersed in the solution in the crystallizers. The intensity of the insonation is controlled by adjusting the amplitude and hence power of the ultrasonic irradiation.

Ultrasound frequencies of around 20 kHz are generally suitable; frequencies in the range 19-25 kHz are particularly suitable, especially 20 kHz. Lower frequencies than these are generally to be avoided since they may fall within a range audible to the human ear. For a given geometry of flow cell, certain frequencies may be prone to cancellation. Generally this phenomenon may be avoided by modest tuning of the probe frequency. For crystallizers having a typical volume of 750 mL, ultrasound power in the range 5-500 W preferably 10-100 W e.g. 20 W with typical power/probe area ratios of 1-80 W/cm² may be suitable although there is an increasing risk of erosion at the face of the acoustic horn as the power density increases; in general smaller particles are obtainable using higher power. Low power/probe area ratios are preferred e.g. in the range 2-30 W/cm², especially 2-20 W/cm². The ultrasound power input is controlled by varying the amplitude of oscillation. For higher or lower crystallizer volumes, the ultrasound power intensity would be adjusted appropriately. An amplifying horn may be used to increase a transducer amplitude of typically 1-12 μm peak-peak to 5-30 μm peak-peak amplitude at the tip of the horn. Conversely a negative gain horn may be used to deliver additional ultrasonic power whilst maintaining a low power density reducing the tendency for erosion of the horn face. Where more than one crystallizer is used ultrasound is preferably deployed in each crystallizer.

The solution of the substance in a liquid solvent and the liquid anti-solvent for said substance are preferably contained in first and second reservoirs adapted for fluid connection with the inlet ports of the flow cell. Desirably the means for delivering the contents of the reservoirs to the flow cell via the inlet ports, at independently controlled flow rates, comprises one or more pumps. Preferably a pump will be provided for each of the reservoirs. A range of pumps are available and may be suitable for the apparatus according to the invention. The pump may, for example, be a gear pump or a peristaltic pump.

The contents of the reservoirs may be delivered to the flow cell at a range of flow rates which will be selected and optimised according to the nature of the substance, the solvent for the substance, the anti-solvent and the power and frequency of the source of ultrasonic radiation. The solubility of the substance in the solvent relative to the anti-solvent is one particular variable. The higher the concentration of substance in solvent, the lower may be the flow rate of anti-solvent relative to the solvent solution. Usually the flow rate of the anti-solvent will exceed that of the solvent solution, the ratio of flow of anti-solvent to solvent typically being 1:1 to 5:1. A ratio of 2:1 is, for example, particularly suitable. This ratio relates to the combined flow to the crystallizer wherein splitting the heptane flow to balance the amount crystallized in each vessel is desirable. The proportion of the heptane anti-solvent fed to the first crystallizer should be less than that to the second and typically the volume flow to the first crystallizer would be less than the flow rate of the solution of drug substance in MEK/MIBK. The balance of the heptane flows into the second crystallizer.

Typically flow rates of solvent solution will be in the range of 0.1 to 100 ml/min especially 0.25 to 4 ml/min at lab scale, or especially 50 to 100 ml/min at pilot scale. Typical flow rates of anti-solvent will be in the range of 0.2 to 200 ml/min especially 0.5 to 8 ml/min at lab scale, or 100 to 200 ml/min at pilot scale.

The diameter of the inlet and the outlet ports may, for example, be in the range 0.5-10 mm, depending on scale and flow-rate, typically 1-5 mm.

The velocity of the flow from the inlet ports may be in the range 0.0002 to 10 m/s e.g. 0.001 to 5 m/s, preferably 0.002 to 2 m/s.

By definition all crystallizing systems are vulnerable to encrustation since the vessel walls are in contact with a supersaturated solution which contains growing crystals. In a continuous crystallization process the rate of formation of encrustation is often the limiting factor in determining the duration of operation between cleaning cycles. There are a number of strategies which can serve to limit encrustation:
operation at a modest level of supersaturation
operation in multiple stages rather than as a single stage process where the all the supersaturation is generated in one vessel
operation with a high crystal surface area reduces the operational supersaturation
provision of good mixing to ensure particles are suspended throughout the supersaturated solution
supersaturation is distributed uniformly throughout the crystallizer irrespective of where it is generated

All of these approaches may be built into the process design for this continuous crystallization, however, encrustation may still be encountered during prolonged operation of the continuous crystallization. The duration of experiments carried out to date by the Inventors indicate that the problem may be well controlled by appropriate selection of conditions (e.g. only 1-2% of the product encrustation after 300 hours of operation).

A feature of the process as described herein which is different from that described in WO00/38811 is that precipitation is not instantaneous on mixing the feed streams but occurs more slowly as supersaturation is generated. The presence of ultrasound allows the nucleation rate to be manipulated to adjust the product particle size. The particle size is also strongly influenced by the residence time in this continuous process.

The flow cells may be manufactured from a range of conventional materials, however these will preferably be selected so as to be unreactive with the substance, the solvent and the anti-solvent, and not affected by the presence of the ultrasound field. The flow cell may be of any suitable size, whether of a size suitable for bench-scale preparation, industrial pilot scale preparation or industrial manufacturing scale. Industrial manufacturing scale production may be achieved by the use of multiple pilot-scale systems. Substance throughputs are a function of the substance, the concentration and the flow rates. However for the purposes of illustration exemplary throughputs of certain substances would be as indicated in the examples.

The process and apparatus according to the invention is particularly useful for the production of crystalline particles in the form of triangular plates having a longest edge length in the range 5-25 μm, more particularly less than 10 μm e.g. around 5 μm.

The product particle size may be controlled by adjusting process parameters, in particular residence time and ultrasonic power. Longer residence time favours smaller particles and higher ultrasound power also favours smaller particles.

The inlet ports should be placed such that newly introduced material is predominantly mixed with the bulk material in the vessel and short circuiting is avoided thus ensuring that the feed is not immediately lost through the outlet. Particle size may also be controlled by solution concentration and antisolvent ratio.

For the generation of small particles by the process according to the invention, it is preferred that the difference between the dissolution properties of the solvent and anti-solvent be as great as possible. For reasons of industrial efficiency (particularly in order to reduce the throughput volumes of liquid) it is preferred to use concentrations of substance in solvent which are as high as possible. Nevertheless the solutions must be stable and not prone to crystallization before discharge into the continuous flow cell. With this end in mind, it may be preferred to use the solution of the substance in the solvent at elevated temperature. The reservoir for the solution may also be provided with a vessel jacket to aid temperature control. It may also be preferable to cool the anti-solvent.

In order to improve temperature control and prevent premature precipitation of the dissolved substance in the lines it will generally be desired to heat-trace the fluid pipework and associated pumps, valves etc. It may be preferred to prime the pumps and pipework by pumping heated or cooled solvent or anti-solvent through the appropriate sections, particularly when the dissolved substance is close to its solubility limit. It may also be preferred to prime the flow cell with pure solvent or anti-solvent. Maintaining control of the solution temperature prevents the possibility of the substance crystallising in the flow cell inlet before being mixed with anti-solvent.

The multiple, e.g. twin, twin crystallizer described above has specific advantages over continuous flow cell crystallizers known in the prior art, in particular it avoids the loss of crystalline product by deposition on the internal surfaces of the flow cell, e.g. on the vessel walls, stirrer (if employed) and ultrasound probe head (if employed), which may occur in conventional flow cells particularly when aggressive crystallization conditions such as high ultrasound amplitude and high antisolvent ratios are used in an attempt to obtain maximum crystallization of the desired substance from the solution. As such, the twin crystallizer may also be useful for the production of crystalline particles other than the particles of the invention.

Thus according to a further aspect of the invention there is provided an apparatus adapted to prepare crystalline particles of a substance which comprises:
(i) a first reservoir adapted to contain said substance dissolved in a liquid solvent;
(ii) a second reservoir adapted to contain liquid anti-solvent for said substance which is miscible with the liquid solvent;
(iii) a first mixing chamber having first and second inlet ports, an outlet port and one or more sources of ultrasonic radiation;
(iv) a second mixing chamber having a first inlet port adapted for fluid connection with the outlet port of the first mixing chamber such that liquid exiting the first mixing chamber flows into the second mixing chamber, a second inlet port adapted for fluid connection with the antisolvent reservoir, an outlet port and one or more sources of ultrasonic radiation;
(v) means for delivering the contents of the first reservoir to the first mixing chamber via the first inlet port, and means for delivering the contents of the second reservoir to the first and second mixing chambers via the second inlet ports; and
(vi) means for collecting particles suspended in the liquid discharged from the outlet port of the second mixing chamber.

Omitting ultrasound from both crystallizers is undesired since it leads to reduced yield of crystal (in some cases no crystals may be produced). Omitting ultrasound from the second crystallizer may lead to undesirable agglomeration of crystals.

The contents of the first and second reservoirs are preferably deliverable to the mixing chambers at independent controlled flow rates.

Particles suspended in the liquid discharged from the outlet of the second flow cell may be collected by means of one of a number of conventional particle capturing techniques e.g. filtration, centrifugation, freeze drying or spray drying.

In respect of filtration means, a wide range of suitable filters are known to persons skilled in the art. Examples of filters include sinters (e.g. glass sinters), fibre filters (e.g. paper and nitrocellulose filters) and membrane filters.

In order to reduce the incidence of undesirable “bridging” between particles during harvesting we have found that it is preferable to flush out any residual solvent used for dissolution by thoroughly washing the filter cake with an anti-solvent for the substance. Preferably the anti-solvent will be the same anti-solvent that is used in the crystallization process.

Alternatively the slurry of crystalline particles which outputs from the second flow cell may first optionally be concentrated by passage through a cross flow filtration apparatus and then may be isolated using spray drying or freeze drying technology.

The particles of the invention produced as described above are preferably isolated by filtration and then washed with a mixture of MIBK, MEK and heptane (preferably of composition matched to that of the mother liquor from which the product is crystallized) to remove chemical impurities in the mother liquors and then with heptane to remove the MEK and MIBK to reduce the risk of the particles granulating together during drying. This washing process delivers predominantly free flowing individual crystals of the unsolvated Form 1 polymorph of the compound of formula (I), when the resulting solvent wet material is dried.

According to a further aspect of the invention there is provided a crystalline particle of unsolvated Form 1 polymorph of the compound of formula (I) wherein the crystalline particle is in the form of substantially triangular plates or triangular plates obtainable by a process as described above.

According to a further aspect of the invention there is provided a population of crystalline particles of unsolvated Form 1 polymorph of the compound of formula (I) wherein the crystalline particles are in the form of substantially triangular plates or triangular plates which when dried are free flowing and easily dispersed as individual primary particles when obtained by a process as described above.

According to a further aspect of the invention there is provided a population of crystalline particles of unsolvated Form 1 polymorph of the compound of formula (I) wherein the crystalline particles are in the form of substantially triangular plates or triangular plates obtainable by a process as described above.

According to a further aspect of the invention there is provided a population of crystalline particles of unsolvated Form 1 polymorph of the compound of formula (I) wherein the crystalline particles are in the form of substantially triangular plates or triangular plates obtained by a process as described above.

The particles of the invention and compositions thereof have potentially beneficial anti-inflammatory or anti-allergic effects, particularly upon topical administration, demonstrated by, for example, the ability to bind to the glucocorticoid receptor and to illicit a response via that receptor, with long acting effect. Hence, the particles of the invention and compositions thereof are useful in the treatment of inflammatory and/or allergic disorders, especially in once-per-day therapy.

Examples of disease states in which the particles of the invention and compositions thereof have utility include skin diseases such as eczema, psoriasis, allergic dermatitis, neurodermatitis, pruritis and hypersensitivity reactions; inflammatory conditions of the nose, throat or lungs such as asthma (including allergen-induced asthmatic reactions), rhinitis (including hayfever), nasal polyps, chronic obstructive pulmonary disease, interstitial lung disease, and fibrosis; inflammatory bowel conditions such as ulcerative colitis and Crohn's disease; and auto-immune diseases such as rheumatoid arthritis.

The particles of the invention may also have use in the treatment of conjunctiva and conjunctivitis.

The particles of the invention are expected to be most useful in the treatment of inflammatory disorders of the respiratory tract e.g. asthma, COPD and rhinitis particularly asthma and rhinitis.

It will be appreciated by those skilled in the art that reference herein to treatment extends to prophylaxis as well as the treatment of established conditions.

As mentioned above, the particles of the invention are useful in human or veterinary medicine, in particular as an anti-inflammatory and anti-allergic agent.

There is thus provided as a further aspect of the invention the particles of the invention for use in human or veterinary medicine, particularly in the treatment of patients with inflammatory and/or allergic conditions, especially for treatment once-per-day.

According to another aspect of the invention, there is provided the use of the particles of the invention for the manufacture of a medicament for the treatment of patients with inflammatory and/or allergic conditions, especially for treatment once-per-day.

In a further or alternative aspect, there is provided a method for the treatment of a human or animal subject with an inflammatory and/or allergic condition, which method comprises administering to said human or animal subject an effective amount of the particles of the invention, especially for administration once-per-day.

The particles of the invention may be formulated for administration in any convenient way, and the invention therefore also includes within its scope pharmaceutical compositions comprising the particles of the invention together, if desirable, in admixture with one or more physiologically acceptable diluents or carriers. Pharmaceutical compositions suitable for once-per-day administration are of particular interest.

The particles of the invention may, for example, be formulated for nasal, oral, buccal, sublingual, parenteral, local or rectal administration, especially local administration.

Local administration as used herein, includes administration by insufflation and inhalation. Examples of various types of preparation for local administration include ointments, lotions, creams, gels, foams, preparations for delivery by transdermal patches, powders, sprays, aerosols, capsules or cartridges for use in an inhaler or insufflator or drops (e.g. eye or nose drops), solutions/suspensions for nebulisation, suppositories, pessaries, retention enemas and chewable or suckable tablets or pellets (e.g. for the treatment of aphthous ulcers) or liposome or microencapsulation preparations.

Advantageously compositions for topical administration to the lung include dry powder compositions and spray compositions.

Dry powder compositions for topical delivery to the lung by inhalation may, for example, be presented in capsules and cartridges for use in an inhaler or insufflator of, for example, gelatine. Formulations generally contain a powder mix for inhalation of the particles of the invention and a suitable powder base (carrier substance) such as lactose or starch. Use of lactose is preferred. When an excipient such as lactose is employed, generally, the particle size of the excipient will be much greater than the particles of the invention. When the excipient is lactose it will typically be present as milled lactose, wherein not more than 85% of lactose particles will have a mass median diameter (MMD) of 60-90 μm and not less than 15% will have a MMD of less than 15 μm. Each capsule or cartridge may generally contain between 20 ∥g-10 mg of the particles of the invention in a pharmaceutical composition optionally in combination with another therapeutically active ingredient. Alternatively, the pharmaceutical compositions may be presented without excipients. Packaging of the formulation may be suitable for unit dose or multi-dose delivery. In the case of multi-dose delivery, the formulation can be pre-metered (e.g. as in Diskus, see GB 2242134 or Diskhaler, see GB 2178965, 2129691 and 2169265) or metered in use (e.g. as in Turbuhaler, see EP 069715). An example of a unit-dose device is Rotahaler (see GB 2064336). The Diskus inhalation device comprises an elongated strip formed from a base sheet having a plurality of recesses spaced along its length and a lid sheet hermetically but peelably sealed thereto to define a plurality of containers, each container having therein an inhalable formulation containing a pharmaceutical composition of the invention preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

Pharmaceutical formulations which are non-pressurised and adapted to be administered as a dry powder topically to the lung via the buccal cavity (especially those which are free of excipient or are formulated with a diluent or carrier such as lactose or starch, most especially lactose) are of particular interest.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the particles of the invention optionally in combination with another therapeutically active ingredient and a suitable propellant such as a fluorocarbon or hydrogen-containing chlorofluorocarbon or mixtures thereof, particularly hydrofluoroalkanes, especially 1,1,1,2-tetrafluoroethane, 1,1,1,2,3,3,3-heptafluoro-n-propane or a mixture thereof. The aerosol composition may optionally contain additional formulation excipients well known in the art such as surfactants e.g. oleic acid or lecithin and cosolvents e.g. ethanol. One example formulation is excipient free and consists essentially of (e.g. consists of) the particles of the invention (optionally together with a further active ingredient) and a propellant selected from 1,1,1,2-tetrafluoroethane, 1,1,1,2,3,3,3-heptafluoro-n-propane and mixture thereof. Another example formulation comprises particles of the invention, a propellant selected from 1,1,1,2-tetrafluoroethane, 1,1,1,2,3,3,3-heptafluoro-n-propane and mixture thereof and a suspending agent which is soluble in the propellant e.g. an oligolactic acid or derivative thereof as described in International Patent Application WO 94/21229. The preferred propellant is 1,1,1,2-tetrafluoroethane. Pressurised formulations will generally be retained in a canister (e.g. an aluminium canister) closed with a valve (e.g. a metering valve) and fitted into an actuator provided with a mouthpiece.

Formulations for administration topically to the nose (e.g. for the treatment of rhinitis) include pressurised aerosol formulations and aqueous formulations administered to the nose by pressurised pump. Formulations which are non-pressurised and adapted to be administered topically to the nasal cavity are of particular interest. The formulation preferably contains water as the diluent or carrier for this purpose. Aqueous formulations for administration to the lung or nose may be provided with conventional excipients such as buffering agents, tonicity modifying agents and the like. Aqueous formulations may also be administered to the nose by nebulisation.

Other possible presentations include the following:

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

If appropriate, the formulations of the invention may be buffered by the addition of suitable buffering agents.

The proportion of the particles of the invention in the pharmaceutical compositions according to the invention depends on the precise type of formulation to be prepared but will generally be within the range of from 0.001 to 10% by weight. Generally, however for most types of preparations advantageously the proportion used will be within the range of from 0.005 to 1% and preferably 0.01 to 0.5%. However, in powders for inhalation or insufflation the proportion used will usually be within the range of from 0.1 to 5%.

Aerosol formulations are preferably arranged so that each metered dose or “puff” of aerosol contains 1 μg-2000 μg, e.g. 20 μg-2000 μg, preferably about 20 μg-500 μg, of the particles of the invention optionally in combination with another therapeutically active ingredient. Administration may be once daily or several times daily, for example 2, 3, 4 or 8 times, giving for example 1, 2 or 3 doses each time. Preferably the pharmaceutical composition of the invention is delivered once or twice daily. The overall daily dose with an aerosol will typically be within the range 10 μg-10 mg e.g. 100 μg-10 mg preferably, 200 μg-2000 μg.

Topical preparations may be administered by one or more applications per day to the affected area; over skin areas occlusive dressings may advantageously be used. Continuous or prolonged delivery may be achieved by an adhesive reservoir system.

For internal administration the particles of the invention may, for example, be formulated in conventional manner for oral, parenteral or rectal administration. Formulations for oral administration include syrups, elixirs, powders, granules, tablets and capsules which typically contain conventional excipients such as binding agents, fillers, lubricants, disintegrants, wetting agents, suspending agents, emulsifying agents, preservatives, buffer salts, flavouring, colouring and/or sweetening agents as appropriate. Dosage unit forms are, however, preferred as described below.

Preferred forms of preparation for internal administration are dosage unit forms i.e. tablets and capsules. Such dosage unit forms contain from 0.1 mg to 20 mg preferably from 2.5 to 10 mg of the particles of the invention.

The particles of the invention may in general be given by internal administration in cases where systemic adreno-cortical therapy is indicated.

In general terms preparations, for internal administration may contain from 0.05 to 10% of the particles of the invention dependent upon the type of preparation involved. The daily dose may vary from 0.1 mg to 60 mg, e.g. 5-30 mg, dependent on the condition being treated, and the duration of treatment desired.

Slow release or enteric coated formulations may be advantageous, particularly for the treatment of inflammatory bowel disorders.

Since the compound of formula (I) is long-acting, preferably the pharmaceutical compositions of the invention will be delivered once-per-day and the dose will be selected so that the compound has a therapeutic effect in the treatment of respiratory disorders (e.g. asthma or COPD, particularly asthma) over 24 hours or more.

The pharmaceutical compositions according to the invention may also be used in combination with another therapeutically active agent, for example, a β₂ adrenoreceptor agonist, an anti-histamine or an anti-allergic. The invention thus provides, in a further aspect, a combination comprising the particles of the invention together with another therapeutically active agent, for example a β₂-adrenoreceptor agonist, an anti-histamine or an anti-allergic.

Examples of β₂-adrenoreceptor agonists include salmeterol (e.g. as racemate or a single enantiomer such as the R-enantiomer or the S-enantiomer), salbutamol (e.g. as racemate or a single enantiomer such as the R-enantiomer), formoterol (e.g. as racemate or a single diastereomer such as the R,R-enantiomer), salmefamol, fenoterol, carmoterol, etanterol, naminterol, clenbuterol, pirbuterol, flerbuterol, reproterol, bambuterol, indacaterol or terbutaline and salts thereof, for example the xinafoate(1-hydroxy-2-naphthalenecarboxylate) salt of salmeterol, the sulfate salt or free base of salbutamol or the fumarate salt of formoterol. Long-acting β₂-adrenoreceptor agonists are preferred, for example, compounds which provide effective bronchodilation for about 12 hours or longer, are preferred.

Other β₂-adrenoreceptor agonists include those described in WO 02/066422, WO 02/070490 WO 02/076933, WO 03/024439, WO 03/072539, WO 03/091204, WO 04/016578, WO 2004/022547, WO 2004/037807, WO 2004/037773, WO 2004/037768, WO 2004/039762, WO 2004/039766, WO01/42193 and WO03/042160.

Particular β₂-adrenoreceptor agonists include:

3-(4-{[6-({(2R)-2-hydroxy-2-[4-hydroxy-3-(hydroxymethyl)phenyl]ethyl}amino)hexyl]oxy}butyl)benzenesulfonamide;

3-(3-{[7-({(2R)-2-hydroxy-2-[4-hydroxy-3-hydroxymethyl)phenyl]ethyl}-amino)heptyl]oxy}propyl)benzenesulfonamide;

4-{(1R)-2-[(6-{2-[(2,6-dichlorobenzyl)oxy]ethoxy}hexyl)amino]-1-hydroxyethyl}-2-(hydroxymethyl)phenol;

4-{(1R)-2-[(6-{4-[3-(cyclopentylsulfonyl)phenyl]butoxy}hexyl)amino]-1-hydroxyethyl}-2-(hydroxymethyl)phenol;

N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;

N-{2-[4-(3-phenyl-4-methoxyphenyl)aminophenyl]ethyl}-2-hydroxy-2-(8-hydroxy-2(1H)-quinolinon-5-yl)ethylamine; and

5-[(R)-2-(2-{4-[4-(2-amino-2-methyl-propoxy)-phenylamino]-phenyl}-ethylamino)-1-hydroxy-ethyl]-8-hydroxy-1H-quinolin-2-one;

and pharmaceutically acceptable salts thereof.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Since the compound of formula (I) is long-acting, preferably the pharmaceutical compositions comprising the particles of the invention and the long-acting β₂-adrenoreceptor agonists will be delivered once-per-day and the dose of each will be selected so that the pharmaceutical composition has a therapeutic effect in the treatment of respiratory disorders effect (e.g. in the treatment of asthma or COPD, particularly asthma) over 24 hours or more.

Examples of anti-histamines include methapyrilene or loratadine.

Other suitable combinations include, for example, other anti-inflammatory agents e.g. NSAIDs (e.g. sodium cromoglycate, nedocromil sodium, PDE4 inhibitors, leukotriene antagonists, iNOS inhibitors, tryptase and elastase inhibitors, beta-2 integrin antagonists and adenosine 2a agonists) or antiinfective agents (e.g. antibiotics and antivirals).

Also of particular interest is use of the particles of the invention in combination with a phosphodiesterase 4 (PDE4) inhibitor e.g. cilomilast or a salt thereof.

The combination referred to above may conveniently be presented for use in the form of a pharmaceutical formulation and thus pharmaceutical formulations comprising a combination as defined above together with a physiologically acceptable diluent or carrier represent a further aspect of the invention.

The particles of the invention in combination with another therapeutically active ingredient as described above may be formulated for administration in any convenient way, and the invention therefore also includes within its scope pharmaceutical formulations comprising the particles of the invention in combination with another therapeutically active ingredient together, if desirable, in admixture with one or more physiologically acceptable diluents or carriers. The preferred route of administration for inflammatory disorders of the respiratory tract will generally be administration by inhalation.

Further, there is provided a process for the preparation of such pharmaceutical compositions which comprises mixing the ingredients.

Therapeutic agent combinations may be in any form, for example combinations may comprise a single dose containing separate particles of individual therapeutics, and optionally excipient material(s), alternatively, multiple therapeutics may be formed into individual multicomponent particles, formed for example by coprecipitation, and optionally containing excipient material(s).

The individual compounds of such combinations may be administered either sequentially in separate pharmaceutical compositions as well as simultaneously in combined pharmaceutical formulations. Appropriate doses of known therapeutic agents will be readily appreciated by those skilled in the art.

The advantages of the particles of the invention may include that the particles have improved mechanical strength, handling and aerodynamic properties and may be generated in an aerodynamic size which is ready for use without need for mechanical size reduction (e.g. micronisation).

### BRIEF DESCRIPTION OF THE FIGURES

FIG. 1 shows an electron micrograph of particles of the invention produced according to Example 1 (Sample IC)

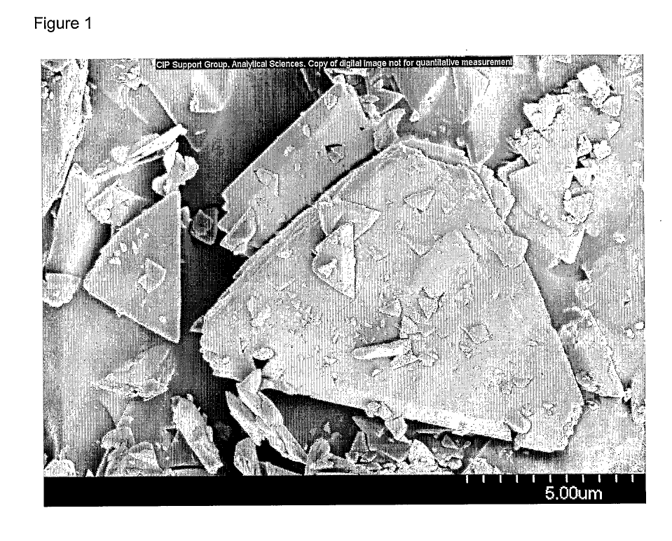

FIG. 2 shows an optical micrograph of particles of the invention produced according to Example 1 (Sample 1A). The bar shown in the top left hand corner of the image indicates a measurement of 20 μm.

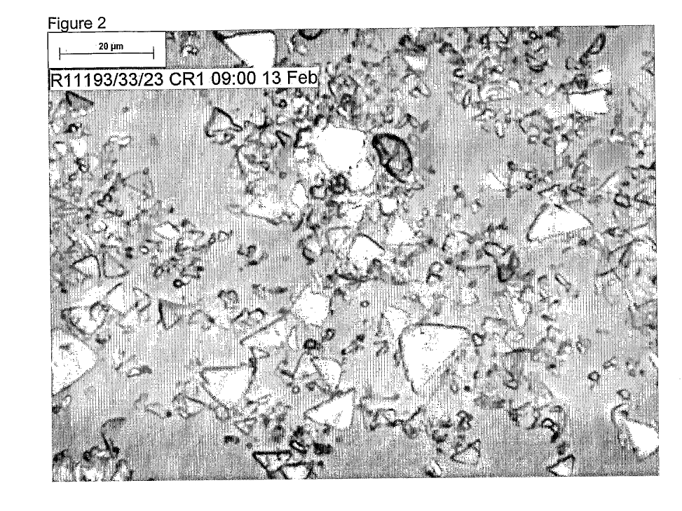

FIG. 3 shows an optical micrograph showing a population of particles of the invention produced according to Example 1 (Sample 1B). The bar shown in the top left hand corner of each image indicates a measurement of 20 μm. Hence the magnification of the upper image is approximately 2.5 times greater than that of the lower image.

FIG. 4 shows the XRPD pattern of a representative sample of particles of the invention (upper trace) as compared with a reference sample of Form 1 of unsolvated 6α,9α-difluoro-17α-(2-furanylcarbonyl)oxy]-11β-hydroxy-16α-methyl-3-oxo-androsta-1,4-diene-17β-carbothioic acid S-fluoromethyl ester obtained previously (lower trace), in both cases determined at ambient temperature (e.g. around 295K).

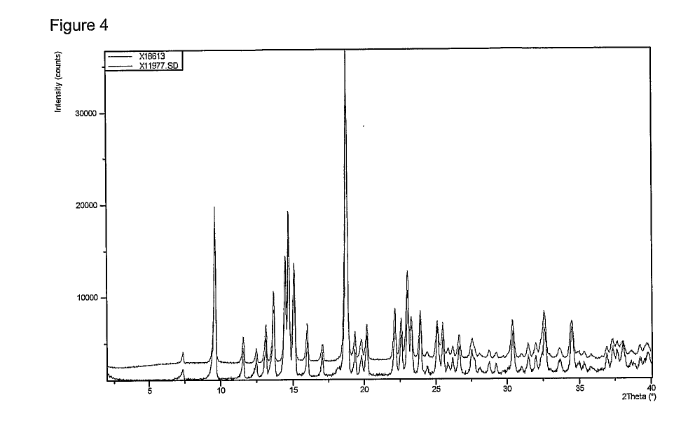

FIG. 5 shows in schematic form a twin crystallizer of use in the production of the particles of the invention

FIG. 6: before and after comparison (optical micrographs) to test the robustness to high shear blending (based on Sample 1C). The bar shown in the top left hand corner of the upper and lower images indicates a measurement of 20 μm and 10 μm respectively. Hence the magnification of the two images is approximately the same.

The following non-limiting Examples illustrate the invention:

### EXAMPLES

General

The XRPD analyses shown in the figures were performed on a PANalytical X'Pert Pro powder diffractometer. The pattern was recorded using the following acquisition conditions: Tube anode: Cu, Start angle: 2.0, End angle: 40.0, Step size: 0.0167, Time per step: 31.75 seconds. XRPD profiles were collected at ambient temperature.

The Scanning Electron Microscopy (SEM) was carried out using a Hitachi S-4700 Field-Emission Scanning Electron Microscope serial number 9323-06. An acceleration voltage of 2.5 kV was used for secondary electron imaging, with instrument magnifications typically within the range of 7000×-10000×

### Example 1

### Twin Crystallizer Description and Operation

Crystallizer Description:

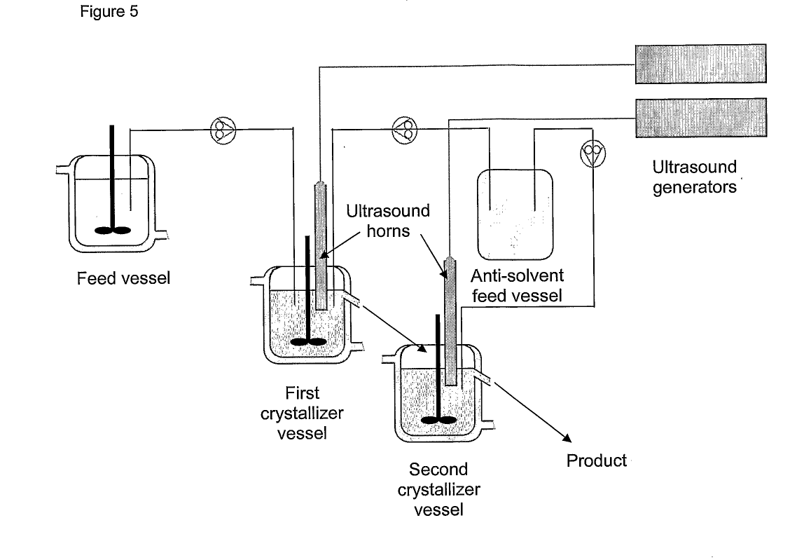

As shown in FIG. 5, the laboratory crystallizer system includes two feed vessels both linked in series to a circulator (Julabo). The first crystallizer, of approximately 60 mL capacity, is linked to a circulator (Huber), the second crystallizer, of approximately 250 mL capacity, is connected to a circulator (Julabo).

The crystallizers are fed from the feed vessels using a peristaltic pump (Watson-Marlow) with modified heated pump head. The feed line is jacketed with an outer tube through which hot liquid is pumped.

The first crystallizer is also fed with heptane from a reservoir which is pumped by a pump (Encynova). There is a similar feed arrangement to the second crystallizer drawing heptane from the same reservoir. The level in the crystallizers is controlled by overflow outlets leading from crystallizer 1 to crystallizer 2 and from crystallizer 2 to the product receiver.

Operation:

The equipment items indicated in FIG. 5 were assembled to form the crystallization system. The circulators providing temperature control for the jacketed components of the crystallization system were adjusted to the desired temperatures and the system allowed to reach thermal equilibrium. The feed vessels were held at elevated temperature typically 90° C., the first crystallizer is typically operated at 30° C. and the second crystallizer is typically operated at 10° C.

n-Heptane was charged to the heptane feed tank. This quantity was selected to provide sufficient antisolvent taking account of the ratio of solution of the compound of formula (I) to antisolvent, the volume of the crystallization vessels selected and the intended duration of the experiment and is typically 10 residence times. It is possible to add further antisolvent during the experiment so avoiding the duration of the experiment being limited by the volume of the antisolvent feed vessels.

A solution of the compound of formula (I) was prepared by dissolution of a sample of the compound of formula (I) in a solvent mixture comprising 90% MIBK by volume and 10% MEK by volume. The quantity of solvent was selected to prepare a solution with a concentration of 1 g of the compound of formula (I) in 14 mL of the MIBK/MEK solvent mixture previously described. In order to achieve dissolution of the compound of formula (I) it was necessary to heat the mixture to a temperature below the boiling point of the mixture when held at normal atmospheric pressure. The quantity of solution of this composition required was determined by the volume of the crystallizer vessels selected and the intended duration of the crystallization e.g. for 10 hours it was 150 mL. It is possible to prepare further feed solution as the process operates so avoiding the duration of the experiment being limited by the volume of the feed vessels. The solution was held in a jacketed feed vessel at an elevated temperature so that the solution could not crystallize on standing.

The pumping rates on the feed solution pump and the antisolvent pumps feeding both the first and second crystallization vessels were set. The pumps were first primed with the solution to be pumped. The pumps could then be calibrated by pumping into measuring cylinders for a suitable period. The experiment was started by commencing feeding of the solution of the compound of formula (I) and the heptane anti-solvent to the first crystallization vessel.

Several start up strategies may be adopted:

The crystallizers may first be charged with a solvent composition which matched that which will be achieved during steady state operation (excluding the contribution of the compound of formula (I)). This solvent mixture in the crystallizer vessels is then slowly displaced as the feed solution of the compound of formula (I) and the heptane antisolvent flow into the crystallizers.

Alternatively, and in the case of this example the crystallizers were charged by initiating the feed flows at the selected rates for the experiments and filling the crystallizer vessels from empty.

Alternatively the crystallizers can be charged from empty by initiating the feed flows at higher rates than those selected for the steady state operation of the crystallization system, reverting to the selected rates for the experiments once the crystallizer vessels are filled to the operating level. Once the tip of the ultrasound horn was submerged the ultrasound generators (Sonic Systems P100) were turned on and the power level adjusted to the selected amplitude and power e.g. amplitude 5 μm, power 16 W using a titanium positive gain acoustic horn with a 9 mm tip diameter.

The product crystals were collected in their mother liquors in a suitable container and isolated.

### Specific Example 1

Feed solutions were prepared in batches based on 100 g of compound of formula (I) being dissolved in a mixture of MIBK 1800 mL and MEK 200 mL this was dissolved by heating and then fed as required to the first stage of a two stage crystallizer system at a rate of approximately 3.6 mL per minute. n-Heptane was also fed to the first crystallizer. with a working volume of around 750 ml. at 1.06 mL per minute, a second feed of n-heptane was added to the second crystallizer, with a working volume of around 830 ml, at a rate of 7.37 mL per minute. At the start of the experiment the crystallizers were charged with a slurry representative of the anticipated steady state of operation. For the first crystallizer the charge comprised 29.1 g of compound of formula (I), 553 mL of a 9:1 MIBK to MEK solvent mixture and 167 mL of heptane. This mixture was prepared as a suspension and charged to the first crystallizer at the start of the experiment. This crystallizer was operated at 30° C. with insonation using a Sonic Systems 500 W ultrasound generator at 20 W and 20,000 kHz. The second crystallizer was charged with product slurry from a previous experiment, this was estimated to comprise; compound of formula (I) 2.2%, MIBK 29.9%, MEK 3.3% and n-heptane 64.6%. This crystallizer was operated at 10° C. with insonation using a Sonic Systems 500 W ultrasound generator at 20 W and 20,000 kHz. The system was operated for 38 hours and 25 minutes. The product Sample 1A was a sample of the slurry of product from the first crystallizer taken at the end of the experiment. The product Sample 1B was a sample of the slurry of product from the second crystallizer taken at the end of the experiment. Sample 1C is the product collected after 25 hours of operation until the end of the experiment.

Total input:

| | |
|---|---|
| Compound of formula (I) | 459.2 g Assay 96.9% |
| MIBK | 6135 g |
| MEK | 689.4 g |
| n-Heptane | 13293 g |

Output:

| | |
|---|---|
| Theory yield | 90% |
| Purity | 98.8% (Sample 1B) |

### Specific Example 2

Feed solutions were prepared in batches based on 100 g of compound of formula (I) being dissolved in a mixture of MIBK 1800 mL and MEK 200 mL this was dissolved by heating and then fed, as required, to the first stage of a two stage crystallizer system at an initial rate of 5.4 mL/min for the first 30 hours and then a reduced rate of 3.6 mL per minute for the remainder of the experiment. For the first 30 hours of the experiment n-heptane was also fed to the first crystallizer at 1.59 mL per minute, a second feed of n-heptane was added to the second crystallizer at a rate of 11.06 mL per minute. For the remainder of the experiment n-heptane was fed to the first crystallizer at 1.06 mL per minute, and the feed rate of n-heptane to the second crystallizer was at a rate of 7.37 mL per minute. The first crystallizer was operated at 30° C. initially with insonation using a Sonic Systems 500 W ultrasound generator at 50 W and 20,000 kHz and then from the 50th hour through to the end of the experiment with insonation at 15 W and 20,000 kHz. The second crystallizer was operated at 10° C. with insonation using a Sonic Systems 500 W ultrasound generator at 50 W and 20,000 kHz.

At the start of the experiment the crystallizers were charged with product slurry from the corresponding first and second crystallizers from the previous example (Specific Example 1 sample 1A and 1B). The system was operated for 91 hours. The product Sample 2A represents the production between hours 28 and 44 and Sample 2B represents the production from hours 44 to 67.

Total input:

| Compound of formula (I) | 1000 g Assay 96.9% |
|---|---|
| MIBK | 14250 g |
| MEK | 1583 g |
| n-Heptane | 34953 g |
| Theory yield | 92% |
| Purity | 98.5% |

### Isolation of continuously crystallized 6α,9α-difluoro-17α-[(2-furanylcarbonyl)oxy]-11β-hydroxy-16α-methyl-3-oxo-androsta-1,4-diene-17β-carbothioic acid S-fluoromethyl ester particles of the Invention

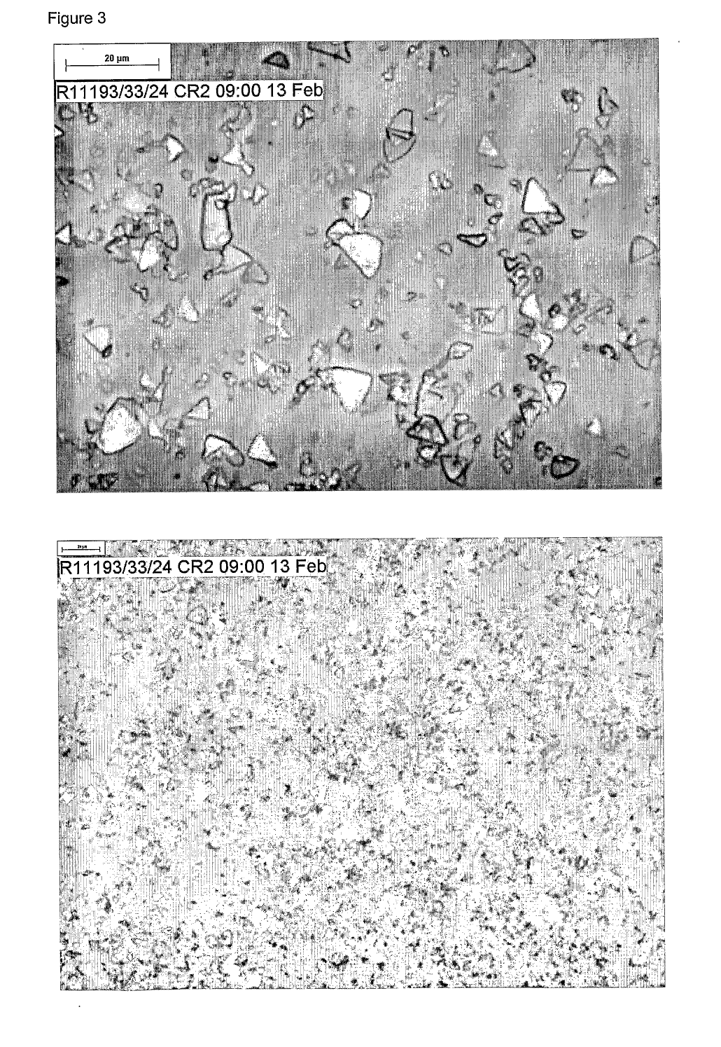

The material was filtered, washed with 2 cake volumes of 9:1:20 v/v MIBK:MEK: n-heptane and then 2 cake volumes of n-heptane. The sample was then re-suspended in n-heptane and re-filtered. The resulting cake was dried in situ. Images at two different magnifications, as shown in FIG. 3, illustrate that the product is easily dispersed and has a characteristic triangular plate like habit.

### Example A

### Dry powder composition containing 6α,9α-difluoro-17α-[(2-furanylcarbonyl)oxy]-11β-hydroxy-16α-methyl-3-oxo-androsta-1,4-diene-17β-carbothioic acid S-fluoromethyl ester particles of the Invention

A dry powder formulation was prepared as follows:

A blend was prepared containing 0.8% w/w 6α,9α-difluoro-17α-[(2-furanylcarbonyl)oxy]-11β-hydroxy-16α-methyl-3-oxo-androsta-1,4-diene-17β-carbothioic acid S-fluoromethyl ester, prepared as particles according to the invention, and 99.2% w/w milled lactose (wherein the mean particle size is of the range 60-90 μm, and not greater than 15% of particles have a MMD of less than 15 μm).

The above composition was blended for 10 minutes at 600 rpm in a 2.5 litre bowl QMM Micromixer. A peelable blister strip containing 14 blisters, each filled with 13 mg of the powder formulation described above was prepared.

Anderson Cascade impaction analysis of this product was performed initially and after 1 month storage at 30° C. and 65% relative humidity as shown in Table 1 below.

TABLE 1

| | Respirable fraction (% of Total Emitted Dose) | | |
|---|---|---|---|
| Storage | | Recrystallized Drug | Recrystallized Drug |
| condition and | Micronised | (Example 2) | (Example 2) |
| Time point | Drug | Sample 2A | Sample 2B |
| Initial | 26.6 | 28.6 | 19.7 |
| 1 month @ | 25.1 | 26.4 | 17.4 |
| 30° C./65% | | | |
| Relative | | | |
| Humidity | | | |

The data shown in Table 1 indicates that a suitable respirable dose at initial and on stability, under the conditions tested, has been achieved and is comparable to an equivalent micronised drug product.

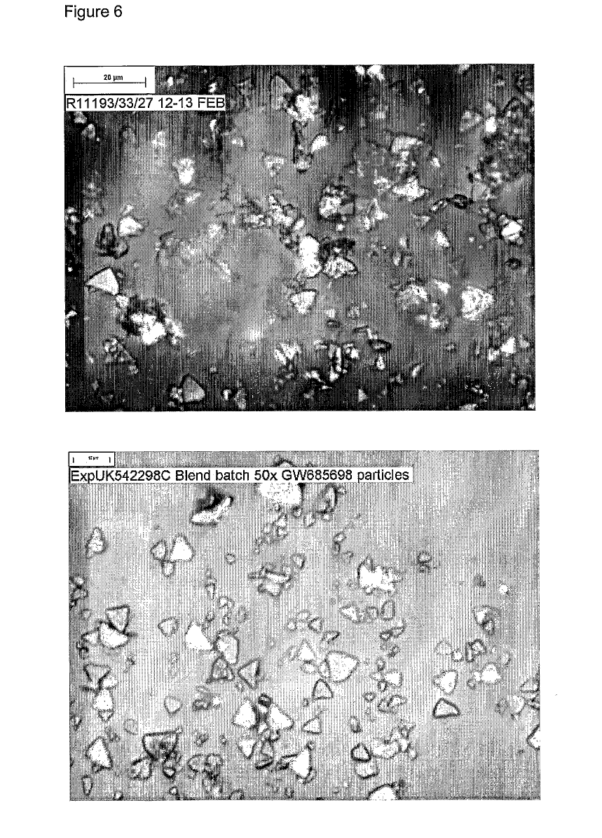

In order to determine the robustness of the product crystals, to the high sheer blending process, a sample of the blend similar to that described above but based on Sample 1C was dispersed on a microscope slide. Water was added to dissolve the lactose leaving the drug substance behind allowing a comparison to be made with drug substance prior to blending. Optical micrographs of the material before and after blending (shown in FIG. 6) reveal little change is particle size and shape suggesting the particles are indeed robust to the blending process.

### Example B

### Dry powder composition containing 6α,9α-difluoro-17α-[(2-furanylcarbonyl)oxy]-11β-hydroxy-16α-methyl-3-oxo-androsta-1,4-diene-17β-carbothioic acid S-fluoromethyl ester particles of the Invention and a long acting β₂-adrenoreceptor agonist

A dry powder formulation may be prepared as follows:

| | |
|---|---|
| 6α,9α-difluoro-17α-[(2-furanylcarbonyl)oxy]-11β- | 0.10 mg |
| hydroxy-16α-methyl-3-oxo-androsta-1,4-diene-17β-carbothioic | |
| acid S-fluoromethyl ester, prepared according to the method of | |
| the invention, MMD of around 3 μm: | |
| Long-acting β₂-adrenoreceptor agonist (micronised | 0.02 mg |
| to a MMD of 3 μm): | |
| milled lactose (wherein not greater than 85% of particles have | 12.5 mg |
| a MMD of 60-90 μm, and not less than 15% of particles have | |
| a MMD of less than 15 μm): | |

A peelable blister strip containing 60 blisters each filled with a formulation as just described may be prepared.

### Example C

### Aerosol formulation containing 6α,9α-difluoro-17α-[(2-furanylcarbonyl)oxy]-11β-hydroxy-16α-methyl-3-oxo-androsta-1,4-diene-17β-carbothioic acid S-fluoromethyl ester particles of the Invention

An aluminium canister may be filled with a formulation as follows:

| | | |
|---|---|---|
| 6α,9α-Difluoro-17α-[(2-furanylcarbonyl)oxy]-11β- | 250 | μg |
| hydroxy-16α-methyl-3-oxo-androsta-1,4-diene-17β- | | |
| carbothioic acid S-fluoromethyl ester, prepared according to | | |
| the method of the invention, MMD of around 3 μm: | | |
| 1,1,1,2-tetrafluoroethane: | to 50 | μl |
| (amounts per actuation) | | |

in a total amount suitable for 120 actuations and the canister may be fitted with a metering valve adapted to dispense 50 μl per actuation.

### Example D

### Aerosol formulation containing 6α,9α-difluoro-17α-[(2-furanylcarbonyl)oxy]-11β-hydroxy-16α-methyl-3-oxo-androsta-1,4-diene-17β-carbothioic acid S-fluoromethyl ester particles of the Invention and a long acting β₂-adrenoreceptor agonist

An aluminium canister may be filled with a formulation as follows:

| | | |
|---|---|---|
| 6α,9α-difluoro-17α-[(2-furanylcarbonyl)oxy]-11β- | 250 | μg |
| hydroxy-16α-methyl-3-oxo-androsta-1,4-diene-17β- | | |
| carbothioic acid S-fluoromethyl, prepared according to | | |
| the method of the invention, MMD of around 3 μm: | | |
| Long-acting β₂-adrenoreceptor agonist | 25 | μg |
| (micronised to a MMD of 3 μm): | | |
| 1,1,1,2-tetrafluoroethane: | to 50 | μl |
| (amounts per actuation) | | |

in a total amount suitable for 120 actuations and the canister may be fitted with a metering valve adapted to dispense 50 μl per actuation.

### Example E

### Nasal formulation containing 6α,9α-difluoro-17α-[(2-furanylcarbonyl)oxy]-11β-hydroxy-16α-methyl-3-oxo-androsta-1,4-diene-17β-carbothioic acid S-fluoromethyl ester particles of the Invention

A formulation for intranasal delivery may be prepared as follows:

| | | |
|---|---|---|
| 6α,9α-difluoro-17α-[(2-furanylcarbonyl)oxy]-11β- | 10 | mg |
| hydroxy-16α-methyl-3-oxo-androsta-1,4-diene-17β- | | |
| carbothioic acid S-fluoromethyl ester, prepared according to | | |
| the method of the invention, MMD of around 3 μm: | | |
| Polysorbate 20 | 0.8 | mg |
| Sorbitan monolaurate | 0.09 | mg |
| Sodium dihydrogen phosphate dihydrate | 94 | mg |
| Dibasic sodium phosphate anhydrous | 17.5 | mg |
| Sodium chloride | 48 | mg |
| Demineralised water | to 10 | ml |

The formulation may be fitted into a spray pump capable of delivering a plurality of metered doses (Valois).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The patents and patent applications described in this application are herein incorporated by reference.

What is claimed is:

1. Crystalline particles of unsolvated Form 1 polymorph of the compound of formula (I): 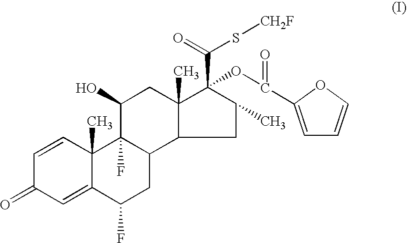
characterised in that the particles are in the form of substantially triangular plates.

2. Crystalline particles as claimed in claim 1
characterised in that the particles are in the form of triangular plates.

3. Crystalline particles as claimed in claim 1 wherein the angles of the triangular faces are approximately 80°, 50° and 50°.

4. Crystalline particles as claimed in claim 1, of size 0.1-0.2 μm×4-5 μm×4-5 μm.

5. A pharmaceutical composition comprising crystalline particles according to claim 1 admixed with a physiologically acceptable diluent or carrier.

6. A pharmaceutical composition according to claim 5 in dry powder form wherein the diluent or carrier is particulate lactose.

7. A method for the treatment of a human or animal subject with an inflammatory and/or allergic condition, which method comprises administering to said human or animal subject an effective amount of crystalline particles according to claim 1.

8. A pharmaceutical composition comprising crystalline particles according to claim 1 in combination with another therapeutically active agent.

9. A pharmaceutical composition according to claim 8 wherein the other therapeutically active ingredient is a long acting β₂-adrenoreceptor agonist.

10. A process for preparing crystalline particles of unsolvated Form 1 polymorph of the compound of formula (I): 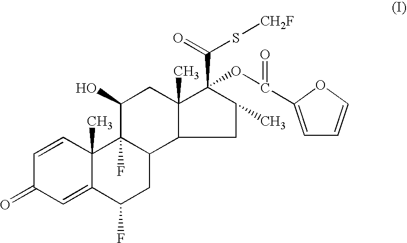
wherein the particles are in the form of substantially triangular plates, which process comprises dissolving the compound of formula (I) in a solvent of methyl-isobutyl-ketone (MIBK) containing between 1 and 15% v/v methyl-ethyl-ketone (MEK), and
producing compound of formula (I) as unsolvated Form 1 polymorph by addition of heptane as anti-solvent.

11. A process for preparing crystalline particles according to claim 10,
wherein the particles are in the form of triangular plates.

12. A process according to claim 10 wherein the solvent contains between 5% and 15% v/v MEK.

13. A process according to claim 12 wherein the solvent contains MIBK and MEK in a ratio of 9:1 v/v.

14. A process according to claim 10 wherein the particles are prepared in a continuous process in the presence of ultrasonic radiation.

15. A population of crystalline particles of unsolvated Form 1 polymorph of the compound of formula (I): 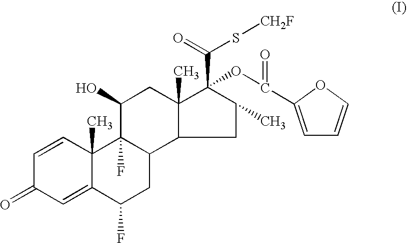
obtainable by the process of claim 14.

16.-17. (canceled)

18. The method of treatment as claimed in claim 7, wherein said effective amount is delivered to the human or animal once-per-day.

19. An apparatus adapted to prepare crystalline particles of a substance which comprises:
(i) a first reservoir adapted to contain said substance dissolved in a liquid solvent;
(ii) a second reservoir adapted to contain liquid anti-solvent for said substance which is miscible with the liquid solvent;
(iii) a first mixing chamber having first and second inlet ports, an outlet port and a source of ultrasonic radiation;
(iv) a second mixing chamber having a first inlet port adapted for fluid connection with the outlet port of the first mixing chamber such that liquid exiting the first mixing chamber flows into the second mixing chamber, a second inlet port adapted for fluid connection with the antisolvent reservoir, an outlet port and a source of ultrasonic radiation;
(v) means for delivering the contents of the first reservoir to the first mixing chamber via the first inlet port, and means for delivering the contents of the second reservoir to the first and second mixing chambers via the second inlet ports; and
(vi) means for collecting particles suspended in the liquid discharged from the outlet port of the second mixing chamber.
